(19) United States
(12) Patent Application Publication
(10) Pub. No.: US 2003/0003446 A1
(43) Pub. Date: Jan. 2, 2003

(54) Method of assessing levels of infectious virus in a biological sample

(76) Inventors: Aristides Lazo, West Newton, MA (US); Asa Ohagen, Brookline, MA (US)

Correspondence Address: MINTZ, LEVIN, COHN, FERRIS, GLOVSKY & POPEO, P.C., One Financial Center, Boston, MA, 02111, US

(21) Appl. No.: 10/165,328

(22) Filed: Jun. 6, 2002

Related U.S. Application Data
(60) Provisional application No. 60/296,289, filed on Jun. 6, 2001.

(51) Int. Cl. C12Q 1/70

(52) U.S. Cl. 435/5

(57) ABSTRACT

Disclosed is a method for identifying levels of an infectious virus in a cell population by comparing the level of a viral gene product in a first population of cells cultured in the presence of the virus with the level of the viral gene product in a second population of cells cultured under conditions that inhibit replication of the virus, e.g., cells that are cultured in the presence of an inhibitor of replication of the virus.

### RELATED APPLICATIONS

This application claims priority to USSN 60/296,289, filed Jun. 6, 2001. The contents of this application are incorporated herein by reference in their entirety.

### STATEMENT OF GOVERNMENT SUPPORT

This invention was made with U.S. Government support under National Institutes of Health/National Cancer Institutes grant. R01 HL66847-01. The government has certain rights in the invention.

### FIELD OF THE INVENTION

The invention relates generally to viruses and more particularly to methods of assessing levels of infectious virus in a biological sample.

### BACKGROUND OF THE INVENTION

When manipulating biological samples containing, or suspected of containing, an infectious virus of interest, it is important to accurately determine the level of infectious virus in the sample. Viral levels are typically determined by examining the levels of a viral gene product, such as a structural protein, in the sample. A biological sample is typically determined to be free of the virus of interest if the level of the viral gene product in the sample is below a pre-selected level.

However, assessing viral levels in this manner can reveal viral gene products that are not associated with infectious virus. For example, the viral gene product may exist freely in the medium containing the biological sample, or the viral gene product may be associated with a virus that is not infectious. In addition, the amount of gene product unassociated with intact virus, or associated with a non-infectious virus, can vary among viruses, and among assays used to detect the virus.

### SUMMARY OF THE INVENTION

The invention is based in part on the discovery of a sensitive and efficient method for determining the levels of infectious virus in a biological sample.

In one aspect, the invention features a method for determining the amount of infectious virus in a cell sample by providing a first population of cells infected with a virus, as well as a second population of cells infected with the virus. The second population of cells further includes an inhibitor of replication of the virus and/or the second population of cells can be cultured in a medium that lacks at least one nutrient, e.g., a growth factor, necessary for replication of the second population of cells. The amount of a viral gene product is then detected in the first population of cells, and the amount of the viral gene product is also detected in the second population of cells. The level of the viral gene product in the first population of cells is compared to the level of the viral product in the second population of cells, thereby determining the amount of infectious virus in the cell sample. For example, subtracting the level of the viral gene product in the second population of cells from the level of the viral gene product in the first population of cells reveals the level of infectious virus in a cell sample.

By “infectious virus” is meant a virus capable of producing a productive infection when introduced into a host cell.

In some embodiments, the first population of cells and the second population of cells are cultured in a medium that allows for replication of the virus in the first population of cells. The levels of the viral gene product in the first population of cells and the second population of cells can be detected at any desired time point. For example, the level of the viral gene product in the first population of cells and the level of the viral gene product in the second population of cells can be detected at least seven days after infecting the first population of cells and the second population of cells with the virus. Levels can also be assessed at one or more subsequent time points, e.g., at 14 or 21 days after infection.

The virus can be any virus for which a viral gene product can be detected, and for which an inhibitor of replication of the virus is known. Examples of suitable viruses include retroviruses, such as lentiviruses. A preferred lentivirus is human imnmunodeficiency virus (HIV). Other preferred viruses are hepatitis B virus and hepatitis C virus.

The viral gene product can be, e.g., a viral nucleic acid (including RNA or DNA) or viral protein. A preferred viral protein is a viral structural protein. When HIV is the virus, a preferred viral protein is the p24 antigen.

A preferred inhibitor is a compound that inhibits an RNA or DNA polymerase encoded by the genome of the virus. For example, when the virus is a retrovirus, the inhibitor preferably inhibits a reverse transcriptase encoded by the genome of the virus. When the virus is HIV, a preferred inhibitor is 2′-Azido-3′deoxythymidine (AZT). When AZT is used, it is preferably present at a concentration of about 20 μM.

The first population of cells and second population of cells are cells in which the virus is capable of initiating and developing a productive infection. When the virus is HIV, the first population and second population of cells are preferably peripheral blood mononuclear cells (PBMC).

In another aspect, the invention provides a method for determining the amount of infectious human immunodeficiency virus in a cell sample by providing a first population of cells infected with a human immunodeficiency virus, as well as providing a second population of cells infected with the human immunodeficiency virus. The second population of cells further includes an inhibitor of a reverse transcriptase encoded by the genome of the human immunodeficiency virus. The amount of a viral protein in the first population of cells and the amount of the viral protein in the second population of cells is detected, and the level of the viral protein in the first population of cells is compared to the level of the viral protein in the second population of cells, thereby determining the amount of infectious virus in the cell sample. A preferred inhibitor is AZT, and a preferred viral protein is p24. The first population of cells and the second population of cells are preferably PBMC.

In a further aspect, the invention features a method for determining the amount of infectious human immunodeficiency virus in a cell sample by providing a first population of -peripheral blood mononuclear cells infected with a human immunodeficiency virus, and providing a second population of peripheral blood mononuclear cells infected with the human immunodeficiency virus. 2′-Azido-3′deoxythymidine (AZT) is also present in the second population of cells. The method also includes detecting the amount of HIV p24 protein in the first population of cells and the amount of the p24 protein in the second population of cells, and then comparing the level of the 24 protein in the first population of cells to the level of the p24 protein the second population of cells, thereby determining the amount of infectious human immunodeficiency virus in the cell sample.

Also featured by the invention is a method for determining the amount of infectious virus in a biological sample. The method includes providing a biological sample known to or suspected of containing an infectious virus and contacting a first population of cells with a first portion of the biological sample under conditions sufficient to allow for introduction of the infectious virus, if present, into the first cell population. The method further includes contacting a second population of cells with a second portion of the biological sample under conditions sufficient to allow for introduction of the infectious virus, if present, into the second cell population, and culturing the first population of cells and the second population of cells in a medium that allows for replication of the virus in the first population of cells. The second population of cells is grown in the presence of a virus replication inhibitor, e.g. AZT or a cell culture medium lacking at least one nutrient necessary for replication of the second cell population. The level of the viral protein in the first population of cells is compared to the level of the viral protein the second population of cells, thereby determining the amount of infectious virus in the biological sample. Preferably, the biological sample is a blood-derived sample.

In some embodiments, the biological sample (such as a blood derived sample) has been contacted with an antiviral agent.

In one aspect, the invention features a method for determining the effectiveness of an antiviral agent by providing a cell sample known to be infected with a virus and culturing a first population of cells from the cell sample, where the first population of cells has been treated with an antiviral agent under predetermined viral inactivating conditions, in a culture medium that allows for replication of the virus. A second population of cells from the cell sample is cultured in a medium that inhibits replication of the virus. A viral protein is detected in the first population of cells and the viral protein is detected in the second population of cells. By comparing the level of the viral protein in the first population of cells to the level of the viral protein the second population of cells, the effectiveness of the antiviral agent under the predetermined viral inactivating conditions can be determined. In some embodiments, the starting infected cell sample may have been treated with an antiviral agent under viral inactivating conditions. Optionally, the level of the viral protein in a third population of cells from the cell sample, where the third cell population is taken prior to any treatment with an antiviral agent and cultured in a medium that allows for replication of the virus, may be compared to the level of the viral protein in the first and second cell populations in determining effectiveness of the antiviral agent.

In one embodiment, the antiviral agent is determined effective where the level of the viral protein in the first cell population is substantially lower than the level of the viral protein in the third cell population where the second cell population is used as a background control, e.g. where the level of the viral protein detected in the first cell population is at least 1 OD log, preferably 2 OD logs, more preferably 3 OD logs lower than the level of viral protein detected in the third cell population. In another embodiment, the antiviral agent is determined effective where the level of viral protein detected in the first cell population is not substantially greater than the level of viral protein detected in the second cell population, e.g. less than 3 OD logs greater than in the second cell population, preferably less than 2 OD logs greater and more preferably less than 1 OD log greater. The medium in which the second cell population is cultured may comprise an inhibitor of virally encoded nucleic acid polymerase, e.g. AZT or may lack a nutrient required for replication of the second cell population e.g. may lack a growth factor or the medium may be physiological buffer. In some embodiments, the effectiveness of the antiviral agent against cell associated virus is determined, e.g. by subculture. In some embodiments, the effectiveness of the antiviral agent against cell free virus is determined, e.g. by subculture.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### DETAILED DESCRIPTION OF THE INVENTION

Assessing levels of infectious virus in methods according to the invention is made possible by identifying the background level of a viral gene product using experimental data in a single assay. Cell populations containing, or suspected of containing, a virus of interest are divided into at least two populations. One population is cultured under conditions allowing for establishment and propagation of a productive infection by the virus. The second population of cells is cultured in a medium that inhibits replication of the virus. After a desired period of time, levels of a viral gene product in the first and second population of cells are determined. The level of infectious virus is determined by subtracting the level of the viral gene product in the first population from the level of the viral gene product from the second population of cells.

By directly identifying the levels of an infectious virus in a cell sample, the methods reduce the need for extensive washing of the cells after infection. This allows for convenient and rapid detection of infectious virus in the sample.

To inhibit replication of the virus, the second population of cells can be cultured in the presence of an inhibitor of replication of the virus; the second population is otherwise cultured under the same conditions as the first population of cells. Alternatively, or in addition, the second population of cells can be cultured in a medium that lacks at least one nutrient, e.g., a growth factor, necessary for replication of the second population of cells. In some embodiments, the second population of cells is cultured in a medium that includes primarily, or exclusively, an isotonic physiological buffer, such as phosphate buffered saline (PBS). For cell-associated viruses, the second population of cells is preferably cultured in a medium (such as PBS)that lacks at least one nutrient.

Examples of viruses that can be tested using the methods of the invention include retroviruses. Retroviruses can include (1) Oncovirinae, including all the oncogenic retroviruses, and several closely related non-oncogenic viruses; (2) Lentivirinae, the “slow retroviruses” such as the human immunodeficiency virus (HIV), simian immunodeficiency virus (SIV), feline immunodeficiency virus (FIV), equine infectious anemia virus (EIAV), CEV and Maedi visna virus(MVV); and (3) Spumavirinae.

When the virus is HIV, preferred antiviral agents are nucleoside and non-nucleoside inhibitors of the HIV reverse transcriptase. Nucleoside inhibitors of HIV reverse transcriptase include, e.g., 2′-Azido-3′deoxythymidine (AZT), 2′,3′-dideoxyinosine (DDI), 2′,3′-dideoxycytidine (DDC), 2′,3′-dideoxy-2′,3′-didehydrothymidine (D4T), and the (−) enantiomer of the nucleoside analogue cytosine-1,3-oxathiolane (3TC).

The methods of the invention can be used to detect levels of infectious in any biological fluid of interest. A preferred fluid is a blood-derived composition. As used herein, the terms “blood-derived compositions” and “blood compositions” are used interchangeably and are meant to include whole blood, blood plasma, blood plasma fractions, blood plasma precipitate (e.g., cryoprecipitate, ethanol precipitate or polyethylene glycol precipitate), blood plasma supernatant (e.g., cryosupernatant, ethanol supernatant or polyethylene glycol supernatant), solvent/detergent (SD) plasma, platelets, intravenous immunoglobulin (IVIG), IgM, purified coagulation factor concentrate, fibrinogen concentrate, or various other compositions which are derived from human or animal. Blood-derived compositions also include purified coagulation factor concentrates (e.g., factor VIII concentrate, factor IX concentrate, fibrinogen concentrate, and the like) prepared by any of various methods common in the art including ion exchange, affinity, gel permeation, and/or hydrophobic chromatography or by differential precipitation.

Additional biological fluids include cerebrospinal fluid, urine, saliva, milk, ductal fluid, tears, or semen.

The methods of the invention can also be used to monitor the effectiveness of antiviral agents, such as ethyleneimine oligomers, e.g. ethyleneimine dimer, trimer, tetramer or derivatives thereof, phenothiazine derivatives, acridine derivatives or riboflavin, in removing and/or inactivating a virus, under viral inactivating conditions, in a biological fluid such as a bodily fluid, e.g. the blood derived compositions discussed above. For example, the method can be used on a viral-containing cell population that has been treated with an antiviral agent such as the anti-viral agents described in U.S. Pat. No. 6,136,586, which is hereby incorporated by reference in its entirety.

As used herein “viral inactivating conditions” refers to the conditions under which viral particles are incubated with antiviral agents, including for example, time of treatment, pH, temperature, salt composition and concentration of antiviral agent.

A preferred antigen for HIV and related viruses is the p24 antigen. This antigen is a viral structural protein and is an important component of infectious HIV particles. The p24 protein is synthesized in the cytoplasm of infected cells and then assembled into virions at the cell plasma membrane, where they normally are released as a part of infectious virions into the cell culture medium. However, if the cell is lysed due to a cytotoxic treatment or cell culture conditions, the p24 proteins will be released into the cell culture medium without being part of the infectious virions. These p24 proteins will detected in, e.g., an ELISA assay as a false-positive result. Moreover, treatment of HIV-infected cells or cell-free various with a reagent targeting exclusively the nucleic acid will inactivate the infectious particles without affecting the structural p24 proteins. These non-infectious (inactivated) viruses and cells may also be recorded as false-positives by the ELISA measurements.

The invention will be further illustrated using the following non-limiting examples.

### EXAMPLE 1

Assessing Levels of Infectious Human Immunodeficiency Virus (HIV) in Peripheral Blood Mononuclear Cells (PBMC)

Levels of infectious human immunodeficiency virus (HIV) in peripheral blood mononuclear cells (PBMC) are determined by measuring levels of the HIV p24 protein in a cell population that has been exposed to HIV.

PBMC are exposed to HIV and divided into two populations. For each population, 1×10⁷/ml PHA-stimulated PBMC are incubated for approximately 16-24 hours at 37° C. One population (the “Background Control” or “BC” population) is additionally treated with AZT at a concentration of 20 μM.

Following incubation, the first and second population of cells are isolated using Ficoll-Hypaque gradients, then plated in replicas of 8 in 24 well-plates at a density of 2×10⁶ cells/well. Viral replication is monitored by measuring absolute OD values generated by an HIV p24 antigen enzyme-linked immunoabsorbent assay (ELISA). Measurements are taken at 7, 14, and 21 days post-infection. The sample is considered positive if levels of HIV p24 antigen in the first population are 3 OD logs greater than the level of the p24 antigen in the BC sample. Negative samples are further tested in subculture controls.

### EXAMPLE 2

Assessment of Effectiveness of a Viral Inactivation Agent in Reducing Levels of Infectious Virus in a Cell Sample.

A high-titer stock of a clinical HIV isolate and stimulated PBMC cultures are used to study the inactivation efficacy of an ethyleneimine oligomer in red blood cell concentrates (RBCC).

HIV is introduced into red blood cell concentrate (RBCC) to a final viral concentration of 10% v/v, followed by the addition of ethyleneimine oligomer to a final concentration of 0.1% v/v and incubation at 22° C. At specified time points, samples are taken, and the ethyleneimine oligomer reaction is stopped by addition of sodium thiosulfate/MOPS to a final concentration of 110 μM each. In separate experiments, infected PBMC instead of cell-free virus are used to spike RBCC. In these studies, the final concentration of cell-associated HIV is 1.0-1.5×10⁷ cells/ml. The spiked RBCC are treated with ethyleneimine oligomer as described above. At specified time points, samples are taken and the ethyleneimine oligomer reaction is stopped by separating the infected cells by Ficoll density gradient centrifugation, followed by extensive washing.

Viral titers of the treated cell-free or cell-associated HIV samples are determined by infecting PHA-P and IL-2 stimulated PBMC, and assaying the culture medium for HIV p24 antigens during a three-week observation period.

To control for HIV p24 present in the culture medium as a result of inactivated virus binding, and/or entering PBMC without establishing a productive infection, parallel infections are performed in the presence of AZT. The HIV p24 levels in the AZT-treated cells provide the background levels for the p24 assay used on the ethyleneimine treated samples during the three-week observation period, as described above. The infectious titers of both cell-free and cell-associated HIV are confirmed by establishing subcultures. Medium from the infected cell cultures is collected on day 14, which is the peak time for virus production, and added to fresh PHA-stimulated PBMC cultures. HIV p24 in the subcultures is measured 7 days and 14 days later.

### EXAMPLE 3

Assessing Levels of Infectious Human Immunodeficiency Virus (HIV) in Peripheral Blood Mononuclear Cells (PBMC) Using a Culture Medium Inhibiting Replication of Said Virus

The level of infectious HIV particles in peripheral blood mononuclear cells is examined by comparing p24 antigen levels in parallel cultures cultured in growth medium or phosphate buffered saline (PBS).

PBMC are infected with HIV. 1:10 serially diluted infected samples are added to indicator cells (2*10⁶ PHA-stimulated PBMC in 24-well plates) and grown in cell culture medium containing RPMI 1640, 20% fetal bovine serum (FBS), and 20 U/ml interleukin-2 (IL2). Virus infectivity is determined by measuring the HIV p24 antigen contents of the cell culture medium by an enzyme-linked imnmunoabsorbent assay (ELISA). Samples for ELISA are collected on days 7, 14, and 21 post infection to increase the assay sensitivity.

To control for the background values of p24 proteins associated with inactivated cells/virus or cell debris, treated cells are plated as described above for the infectivity titration onto indicator cells. However, cells from these control titrations are grown in phosphate buffered saline (PBS) instead of cell culture medium. The absence of nutrients and growth stimulating factors (which are present in the cell culture medium mixture) prevents cell proliferation and virus replication. The p24 proteins present in these control cultures represent the default value of viral proteins without any active virus spread. The p24 values obtained from the control cultures are thus used as cut-off values for scoring results as positive or negative for virus replication.

Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A method for determining the amount of infectious virus in a cell sample, the method comprising:
providing a first population of cells known to or suspected of being infected with a virus;
providing a second population of cells known to or suspected of being infected with said virus, said second population of cells further comprising an inhibitor of replication of said virus;
detecting a viral protein in the first population of cells and said viral protein in the second population of cells; and
comparing the level of the viral protein in the first population of cells to the level of the viral protein the second population of cells,
thereby determining the amount of infectious virus in said cell sample.

2. The method of claim 1, further comprising culturing the first population of cells and the second population of cells in a medium that allows for replication of said virus in said first population of cells.

3. The method of claim 1, wherein the level of the viral protein in the first population of cells and the level of the viral protein in the second population of cells are detected at least seven days after infecting the first population of cells and the second population of cells with said virus.

4. The method of claim 1, wherein said virus is a lentivirus.

5. The method of claim 4, wherein said lentivirus is a human immunodeficiency virus (HIV).

6. The method of claim 4, wherein said viral protein is a structural protein.

7. The method of claim 6, wherein said structural protein is p24 antigen.

8. The method of claim 1, wherein said inhibitor inhibits a RNA or DNA polymerase encoded by the genome of said virus.

9. The method of claim 8, wherein said inhibitor inhibits a reverse transcriptase encoded by the genome of said virus.

10. The method of claim 9, wherein said inhibitor is 2′-Azido-3′deoxythymidine (AZT).

11. The method of claim 10, wherein AZT is present in said medium at a concentration of about 20 μM.

12. The method of claim 5, wherein said first population of cells and second population of cells have been contacted with a viral inactivation agent.

13. The method of claim 5, wherein said first population of cells and second population of cells are peripheral blood mononuclear cells (PBMC).

14. A method for determining the amount of infectious human immunodeficiency virus in a cell sample, the method comprising:
providing a first population of cells infected with a human immunodeficiency virus;
providing a second population of cells infected with said human immunodeficiency virus, said second population of cells further comprising an inhibitor of a reverse transcriptase encoded by the genome of said human immunodeficiency virus;
detecting the amount of a viral protein in the first population of cells and the amount of said viral protein in the second population of cells; and
comparing the level of the viral protein in the first population of cells to the level of the viral protein the second population of cells,
thereby determining the amount of infectious virus in said cell sample.

15. The method of claim 14, wherein said inhibitor is 2′-Azido-3′deoxythymidine (AZT).

16. The method of claim 14, wherein said viral protein is p24.

17. The method of claim 14, wherein said first population of cells and second population of cells comprise peripheral blood mononuclear cells (PBMC).

18. A method for determining the amount of infectious human immunodeficiency virus in a cell sample, the method comprising:
providing a first population of peripheral blood mononuclear cells infected with a human immunodeficiency virus;
providing a second population of peripheral blood mononuclear cells infected with said human immunodeficiency virus, said second population of cells further comprising 2′-Azido3′deoxythymidine (AZT);
detecting the amount of HIV p24 protein in the first population of cells and the amount of said p24 protein in the second population of cells; and
comparing the level of the 24 protein in the first population of cells to the level of the p24 protein the second population of cells,
thereby determining the amount of infectious human immunodeficiency virus in said cell sample.

19. A method for determining amount of a infectious virus in a biological sample, the method comprising:
providing a biological sample known to or suspected of containing an infectious virus;
contacting a first population of cells with a first portion of said biological sample under conditions sufficient to allow for introduction of said infectious virus, if present, into said first cell population;
contacting a second population of cells with a second portion of said biological sample under conditions sufficient to allow for introduction of said infectious virus, if present, into said second cell population;
culturing the first population of cells and the second population of cells in a medium that allows for replication of said virus in said first population of cells;
detecting the amount of a viral protein in the first population of cells and the amount of said viral protein in the second population of cells; and
comparing the level of the viral protein in the first population of cells to the level of the viral protein the second population of cells,
thereby determining the amount of infectious virus in said biological sample.

20. The method of claim 19, wherein said biological sample is a blood-derived sample.

21. The method of claim 19, wherein said biological sample has been contacted with an antiviral agent.

22. The method of claim 20, wherein said blood-derived sample has been contacted with an antiviral agent.

23. A method for determining the amount of infectious virus in a cell sample, the method comprising:
culturing a first population of cells known to be or suspected of being infected with a virus in a medium that allows for replication of said virus;
culturing a second population of cells known to be or suspected of being infected with said virus in a medium that inhibits replication of said virus;
detecting a viral protein in the first population of cells and said viral protein in the second population of cells; and
comparing the level of the viral protein in the first population of cells to the level of the viral protein the second population of cells,
thereby determining the amount of infectious virus in said cell sample.

24. The method of claim 23, wherein the level of the viral protein in the first population of cells and the level of the viral protein in the second population of cells are detected at least seven days after infecting the first population of cells and the second population of cells with said virus.

25. The method of claim 23, wherein said virus is a lentivirus.

26. The method of claim 25, wherein said lentivirus is a human immunodeficiency virus.

27. The method of claim 23, wherein said viral protein is a structural protein.

28. The method of claim 27, wherein said structural protein is p24 antigen.

29. The method of claim 23, wherein said first population of cells and second population of cells comprise peripheral blood mononuclear cells (PBMC).

30. The method of claim 23, wherein said second population of cells is cultured in the presence of an inhibitor of replication of said virus.

31. The method of claim 30, wherein said inhibitor inhibits a RNA or DNA polymerase encoded by the genome of said virus.

32. The method of claim 31, wherein said inhibitor inhibits a reverse transcriptase encoded by the genome of said virus.

33. The method of claim 32, wherein said inhibitor is 2′-Azido-3′deoxythymidine (AZT).

34. The method of claim 33, wherein AZT is present in said medium at a concentration of about 20 μM.

35. The method of claim 23, wherein said second population of cells is cultured in the absence of at least one nutrient necessary for replication of said second population of cells.

36. The method of claim 35, wherein said nutrient is a growth factor.

37. The method of claim 35, wherein said second population of cells is cultured in a medium consisting essentially of a physiological buffer.

38. The method of claim 35, wherein said physiological buffer is phosphate-buffered saline.

39. A method for determining the amount of infectious virus in a cell sample, the method comprising:
culturing a first population of cells known to be or suspected of being infected with a virus in a medium that allows for replication of said virus;
culturing a second population of cells known to be or suspected of being infected with said virus in a medium in the absence of at least one nutrient necessary for replication of said second population of cells, thereby inhibiting replication of said virus;
detecting a viral protein in the first population of cells and said viral protein in the second population of cells; and
comparing the level of the viral protein in the first population of cells to the level of the viral protein the second population of cells,
thereby determining the amount of infectious virus in said cell sample.

40. A method for determining the effectiveness of an antiviral agent, the method comprising:
providing a cell sample known to be infected with a virus;
culturing a first population of cells from the cell sample, where the first population of cells has been treated with an antiviral agent under predetermined viral inactivating conditions, in a culture medium that allows for replication of the virus;
culturing a second population of cells from the cell sample in a culture medium that inhibits replication of the virus;
detecting a viral protein in the first population of cells and said viral protein in the second population of cells; and
comparing the level of the viral protein in the first population of cells to the level of the viral protein the second population of cells,
thereby determining the amount of infectious virus in the first cell population and, thus, the effectiveness of the antiviral agent.

41. The method of claim 40, wherein in the cell sample has been treated with an antiviral agent under viral inactivating conditions.

42. The method of claim 40, wherein the antiviral agent is an phenothiazine derivative, acridine derivative, riboflavin or an ethyleneimine oligomer or derivative thereof.

43. The method of claim 40, wherein the culture medium of the second cell population comprises an inhibitor of RNA or DNA polymerase encoded by the genome of said virus.

44. The method of claim 43, wherein the culture medium of the second cell population comprises an inhibitor of a reverse transcriptase encoded by the genome of said virus.

45. The method of claim 44, wherein said inhibitor is 2′-Azido-3′deoxythymidine (AZT).

46. The method of claim 40, wherein the culture medium of the second population of cells lacks at least one nutrient necessary for replication of said second population of cells.

47. The method of claim 46, wherein said nutrient is a growth factor.

48. The method of claim 47, wherein said second population of cells is cultured in a medium consisting essentially of a physiological buffer.

49. The method of claim 42, wherein the antiviral agent is ethyleneimine dimer, trimer or tetramer.

50. The method of claim 41, further comprising comparing the level of the viral protein from cell free virus of the first population of cells to the level of the viral protein of cell free virus of the second population of cells,
thereby determining the amount of infectious cell free virus in the first cell population and, thus, the effectiveness of the antiviral agent against cell free virus.

51. The method of claim 41, further comprising comparing the level of the viral protein from cell associated virus of the first population of cells to the level of the viral protein of cell associated virus of the second population of cells,
thereby determining the amount of cell associated virus in the first cell population and, thus, the effectiveness of the antiviral agent against cell associated virus.
